(19) United States
(12) Patent Application Publication
(10) Pub. No.: US 2014/0207051 A1
(43) Pub. Date: Jul. 24, 2014

(54) METHOD AND APPARATUS FOR CONTROLLING ULTRASOUND SYSTEM

(71) Applicant: SAMSUNG ELECTRONICS CO., LTD., Suwon-si (KR)

(72) Inventors: Won-chul BANG, Seongnam-si (KR); Ho-taik LEE, Yongin-si (KR); Joon-ho SEO, Seoul (KR)

(73) Assignee: SAMSUNG ELECTRONICS CO., LTD., Suwon-si (KR)

(21) Appl. No.: 14/161,977

(22) Filed: Jan. 23, 2014

(30) Foreign Application Priority Data
Jan. 23, 2013 (KR) 10-2013-0007654

(51) Int. Cl. A61M 37/00

(52) U.S. Cl. CPC A61M 37/0092
USPC 604/22

(57) ABSTRACT

Provided are an ultrasound system and methods that deliver medication through skin by using multiple frequencies. The method to deliver medication through skin include irradiating the skin with ultrasound having a first frequency to cavitate a skin tissue; irradiating the skin with ultrasound having a second frequency, which is lower than the first frequency, to collapse the cavitated tissue; and delivering the medication through the collapsed tissue, wherein a single transducer is configured to produce the ultrasound having the first frequency and the ultrasound having the second frequency.

### CROSS-REFERENCE TO RELATED APPLICATIONS

This application claims the benefit under 35 U.S.C. §119(a) of Korean Patent Application No. 10-2013-0007654, filed on Jan. 23, 2013, in the Korean Intellectual Property Office, the entire disclosure of which is hereby incorporated by reference for all purposes.

### BACKGROUND

1. Field

The following description relates to ultrasound systems and methods of controlling them. The following description also relates to an ultrasound system that delivers medication through the skin by using multiple frequencies and a method of controlling the ultrasound system.

2. Description of Related Art

Generally, ultrasound is out of an audible frequency range of a human ear. The hearing range is on an average between 20-20,000 Hz. Ultrasound has roughly has two biological effects.

The first one is a thermal effect, which is caused when ultrasound propagates in a biological tissue. The acoustic energy is absorbed and transformed into thermal energy, increasing the temperature of the biological tissue, such that if the temperature of the biological tissue is higher than a threshold temperature of about 60° C. or higher, then necrosis occurs in a soft tissue and a blood vessel. A recently commercialized high intensity focused ultrasound (HIFU) knife uses such a thermal effect of ultrasound. HIFU treatment using the HIFU knife is a therapy that irradiates HIFU to a tumor portion to be treated while focusing on the tumor portion to cause focal destruction or necrosis of a tumor tissue, thereby removing and curing the tumor.

The second one is a dynamic effect where a tissue injury occurs due to cavitation. When a biological tissue is exposed to ultrasound of high energy, moisture in a cell is transformed into gas, generating micro-bubbles. If the micro-bubbles grow bigger to a degree to which a resonance phenomenon occurs, they explode generating shock waves of high pressure and damaging peripheral tissues. Ultrasound having such biological effects has been widely used in the medical field for diagnosis and treatment of diseases, and a method of using ultrasound to facilitate transcutaneous medication delivery is called phonophoresis.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect there is provided a method to deliver medication through skin including irradiating the skin with ultrasound having a first frequency to cavitate a skin tissue; irradiating the skin with ultrasound having a second frequency, which is lower than the first frequency, to collapse the cavitated tissue; and delivering the medication through the collapsed tissue, wherein a single transducer is configured to produce the ultrasound having the first frequency and the ultrasound having the second frequency.

The single transducer may comprises multiple elements, a first element that is configured to irradiate the ultrasound having the first frequency and a second element that is configured to irradiate the ultrasound having the second frequency.

The first frequency may be at least three times the second frequency.

The single transducer may be configured to irradiate the skin with the ultrasound having the first frequency and the ultrasound having the second frequency in a time-division manner.

The single transducer may be configured to irradiate the skin with the ultrasound having the first frequency and the ultrasound having the second frequency in a space-division manner.

The single transducer may be a capacitive micromachined ultrasound transducer (cMUT).

Each of the multiple elements may be formed of a piezoelectric material.

The first element and the second element may be arranged in a circular manner in the single transducer.

The first element and the second element may be arranged at random in the single transducer.

The first element and the second element may be arranged in a quadrilateral array in the single transducer.

A phase-array scheme may be used to change a position of irradiation without changing the position of the single transducer.

A time of irradiating the skin with ultrasound having a second frequency and a time of irradiating the skin with ultrasound having a first frequency may be based on at least one of characteristics of the ultrasound system, type of medical treatment, medication, and a molecular weight of the medication.

In another general aspect there is provided an ultrasound system to deliver medication through skin including a first ultrasound module configured to irradiate a skin with ultrasound having a first frequency to cavitate the skin tissue; a second ultrasound module configured to irradiate the skin with ultrasound having a second frequency, which is lower than the first frequency, to collapse the cavitated tissue; and a controller configured to drive the first ultrasound module and the second ultrasound module, wherein the first ultrasound module and the second ultrasound module are disposed in a single transducer.

The controller may drive the first ultrasound module and the second ultrasound module in a time-division manner.

The first ultrasound module and the second ultrasound module may be formed of a crystal material.

The first ultrasound module and the second ultrasound module may be capacitive micromachined ultrasound transducer (cMUT).

The first ultrasound module and the second ultrasound module may be formed of a piezoelectric material.

The first ultrasound module and the second ultrasound module may be arranged in a circular manner in the single transducer.

The first ultrasound module and the second ultrasound module may be arranged at random in the single transducer.

The first ultrasound module and the second ultrasound module may be arranged in a quadrilateral array in the single transducer.

The controller may include a first driving controller and a second driving controller, wherein the first driving controller may be configured to drive the first ultrasound module and the second driving controller may be configured to drive the second ultrasound module.

In another general aspect there is provided an ultrasound system to deliver medication through skin, the ultrasound system including a transducer comprising a first ultrasound module configured to irradiate ultrasound having a first frequency and a second ultrasound module configured to irradiate ultrasound having a second frequency, which is lower than the first frequency; and a controller configured to control the transducer to irradiate a skin with the ultrasound having the first frequency and the ultrasound having the second frequency.

The controller is configured to control the first ultrasound module and the second ultrasound module in a time-division manner to alternately irradiate the skin.

Other features and aspects will be apparent from the following detailed description, the drawings, and the claims.

### BRIEF DESCRIPTION OF THE DRAWINGS

FIG. 1 is a diagram illustrating an example of an ultrasound system.

FIG. 2 is a diagram illustrating an example of an ultrasound system.

FIGS. 3A and 3B are diagrams illustrating examples for comparing a case where medication is delivered using one frequency with a case where medication is delivered using multiple frequencies.

FIGS. 4A and 4B are diagrams illustrating examples of using an ultrasound system.

FIG. 5 is a diagram illustrating an example of a method of controlling an ultrasound system.

FIGS. 6A through 6D are diagrams illustrating examples of a transducer for using multiple frequencies.

FIG. 7 is a diagram illustrating an example of a controller shown in FIG. 2.

FIG. 8 is diagram illustrating an example of a controller shown in FIG. 2.

FIG. 9 is a diagram illustrating an example for describing a method of controlling an ultrasound system.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

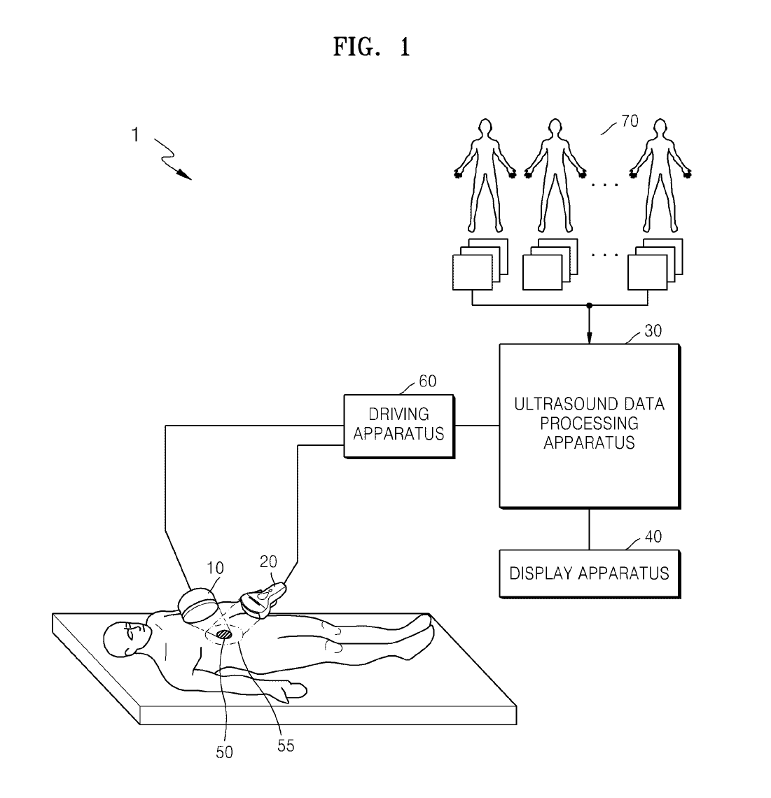

FIG. 1 is a diagram illustrating an example of an ultrasound system 1. Referring to FIG. 1, the ultrasound system 1 includes a treatment ultrasound apparatus 10, a diagnosis ultrasound apparatus 20, an ultrasound data processing apparatus 30, a display apparatus 40, and a driving apparatus 60. In the ultrasound system 1 shown in FIG. 1, only components related to the present example are shown. Thus, those skilled in the art may understand that general components except for components illustrated in FIG. 1 may be further included. For example, the ultrasound system 1 may include an interface unit (not illustrated). The interface unit may be responsible for inputting and outputting input information regarding a user and an image. The interface unit may include a network module for connection to a network and a universal serial bus (USB) host module for forming a data transfer channel with a mobile storage medium, depending on a function of the ultrasound system 1. In addition, the interface unit may include an input/output device such as a mouse, a keyboard, a touch screen, a monitor, a speaker, and a software module for running the input/output device. In addition, the ultrasound system 1 may further include a storage unit (not illustrated) that stores models that are described below. The storage unit may include, for example, a hard disk drive (HDD), a read only memory (ROM), a random access memory (RAM), a flash memory, or a memory card as an ordinary storage medium. In addition, as described below, an external medical image 70, which is an image captured by a medical expert to diagnose a patient's disease may be input to the ultrasound data processing apparatus 30.

For example, as shown in FIG. 1, to treat the tumor in the patient, the treatment ultrasound apparatus 10 of the ultrasound system 1 generates a lesion by irradiating treatment ultrasound onto a treatment target portion 50 of the tumor. The diagnosis ultrasound apparatus 20 of the ultrasound system 1 irradiates diagnosis ultrasound to a peripheral portion 55, which includes the treatment target portion 50 and receives reflected waves. The ultrasound system 1 converts the received reflected waves into an echo signal and acquires ultrasound images based on the echo signal to diagnose whether the treatment has been completed. The lesion means focal destruction or necrosis of a tissue of the treatment target portion 50. The ultrasound system 1 treats the treatment target portion 50 by using the treatment ultrasound apparatus 10 that irradiates the treatment ultrasound onto the treatment target portion 50 of the body of the patient, for example, a portion of the tumor, and monitors treatment results such as the temperature of the treatment target portion 50 by using the diagnosis ultrasound apparatus 20 that irradiates diagnosis ultrasound onto an observation portion.

The treatment ultrasound apparatus 10 may be referred to as a treatment probe. The treatment ultrasound apparatus 10 may irradiate treatment ultrasound to various portions of the body of the patient while moving under the control of the driving apparatus 60. The treatment ultrasound apparatus 10 may irradiate treatment ultrasound to various portions of the body of the patient to change the position of a focus of irradiation of the treatment ultrasound without a change of the position of the treatment ultrasound apparatus 10, that is, while being fixed. The treatment ultrasound apparatus 10 generates treatment ultrasound and irradiates treatment ultrasound to a local tissue of the patient. The treatment ultrasound may be high-intensity focused ultrasound (HIFU) that has sufficient energy for necrosis of the tumor in the patient's body. The treatment ultrasound apparatus 10 corresponds to an apparatus for irradiating HIFU generally known as treatment ultrasound. HIFU is known to those of ordinary skill in the art and thus will not be described in detail. A phase array (PA) scheme may be used where the position of the focus of irradiation of treatment ultrasound is changed in a state where the position of the treatment ultrasound apparatus 10 is fixed.

The diagnosis ultrasound apparatus 20 may be referred to as a diagnosis probe. The diagnosis ultrasound apparatus 20 irradiates diagnosis ultrasound toward an observation portion 55 under control of the driving apparatus 60. The observation portion 55 may have an area that is larger than or equal to the treatment portion 50. The diagnosis ultrasound apparatus 20 receives reflected waves of irradiated diagnosis ultrasound from the diagnosis ultrasound-irradiated portion. The diagnosis ultrasound apparatus 20 may include a piezoelectric transducer. If ultrasound in a range of 2 MHz-18 MHz is delivered from the diagnosis ultrasound apparatus 20 to a particular portion of the patient's body, the ultrasound is partially reflected from layers between different tissues. Ultrasound is reflected in a portion of the body where density change occurs, for example, blood cells in blood plasma and small structures in organs. The reflected ultrasound vibrate the piezoelectric transducer of the diagnosis ultrasound apparatus 20 and the piezoelectric transducer outputs electric pulses corresponding to the vibrations.

The treatment ultrasound apparatus 10 and the diagnosis ultrasound apparatus 20 are shown as independent apparatuses, but they may be implemented as separate modules in one apparatus or may be implemented as one apparatus. Each of the treatment ultrasound apparatus 10 and the diagnosis ultrasound apparatus 20 may also be provided as plural apparatuses as well as a single apparatus. The treatment ultrasound apparatus 10 and the diagnosis ultrasound apparatus 20 may irradiate ultrasound in any direction. For example, while the treatment ultrasound apparatus 10 and the diagnosis ultrasound apparatus 20 are shown irradiating ultrasound from top to bottom in the body of the patient in FIG. 1, they may also irradiate ultrasound in various other directions, for example, from bottom to top in the body of the patient.

The driving apparatus 60 controls the positions of the treatment ultrasound apparatus 10 and the diagnosis ultrasound apparatus 20. The driving apparatus 60 receives position information regarding the treatment target portion 50 from the ultrasound data processing apparatus 30 and controls the position of the treatment ultrasound apparatus 10 such that the treatment ultrasound apparatus 10 accurately irradiates treatment ultrasound to the treatment target portion 50. The driving apparatus 60 receives position information regarding the observation portion from the ultrasound data processing apparatus 30 and controls the position of the diagnosis ultrasound apparatus 20 such that the diagnosis ultrasound apparatus 20 accurately irradiates diagnosis ultrasound to the observation portion and receives reflected waves of the diagnosis ultrasound.

As described above, the ultrasound system 1 monitors a change in the temperature of the observation portion by using the diagnosis ultrasound apparatus 20. In ultrasound treatment using treatment ultrasound such as HIFU, when the HIFU arrives at a portion of the tumor, the temperature of the tumor portion may instantly increase to about 70° C. or higher due to thermal energy of the HIFU. Theoretically, it is known that tissue destruction occurs in 110 msec or less at a temperature of about 60° C. Due to the high temperature, the tissue and blood vessel of the tumor portion are subject to coagulative necrosis.

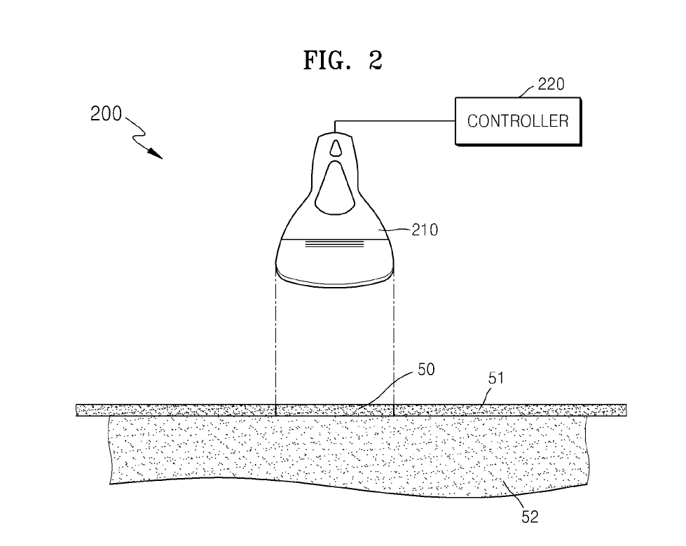

FIG. 2 is a diagram illustrating an example of an ultrasound system 200. Referring to FIG. 2, the ultrasound system 200 includes one transducer 210 and a controller 220 for controlling the transducer 210. The ultrasound system 200 irradiates ultrasound of a particular frequency to the treatment target portion 50 on a skin layer 51 of the patient, thereby delivering medication into a body 52 under the skin together with the irradiated ultrasound. The medication delivered into the body is the medication that is applied together with ultrasound treatment or medication that may directly treat a particular disease of the patient. Therefore, the ultrasound system 200 may be used together with a HIFU system like the ultrasound system 1 shown in FIG. 1, or may be directly used for medication treatment of a particular disease of the patient, such as, for example, a tumor. Thus, the transducer 210 illustrated in FIG. 2 may be the treatment ultrasound apparatus 10 or the diagnosis ultrasound apparatus 20 illustrated in FIG. 1, or may be a part of a medication delivery ultrasound apparatus.

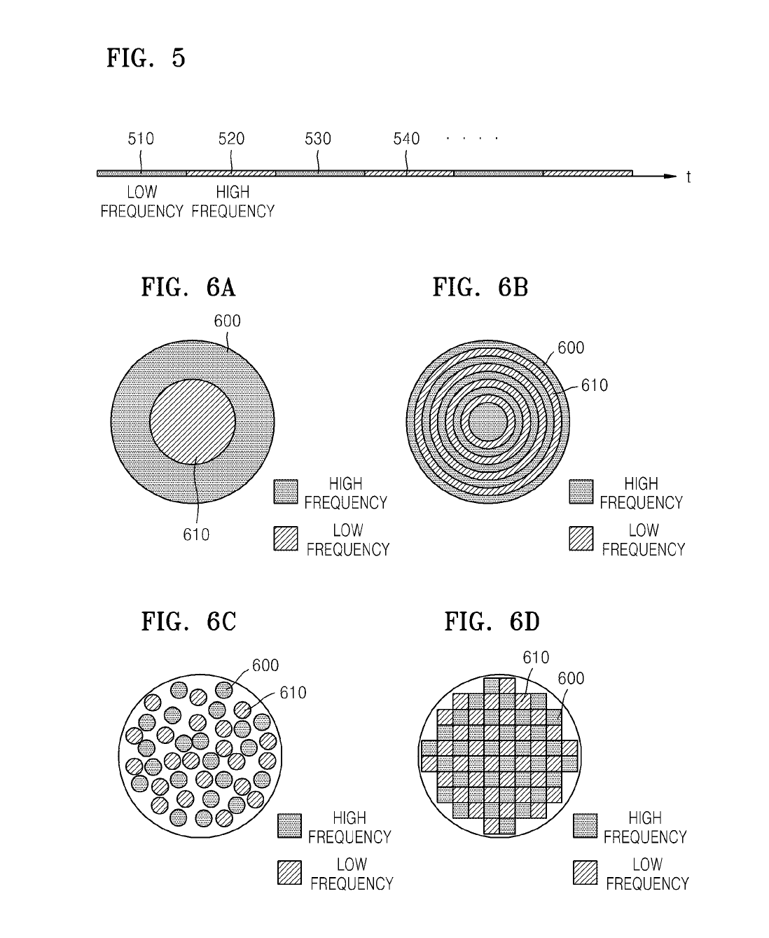

The transducer 210 has a piezoresonator to convert electric energy into ultrasound or to convert ultrasound into electric energy. The transducer 210 may include a plurality of piezoresonators, which may be arranged in various forms. For example, the piezoresonators may be arranged in the transducer 210 may be in an array form, or in an n×m matrix form where several piezoresonators are connected, or a form where the piezoresonators are clustered in a circle. The transducer 210 includes an ultrasound module that generates ultrasound of a high frequency, i.e. of 0.7 Mhz or higher, and an ultrasound module that generates ultrasound of a low frequency, i.e. of 100 kHz or lower. The transducer 210 may be implemented with a material capable of generating ultrasound of a high frequency of 0.7 Mhz or higher and of generating ultrasound of a low frequency of 100 kHz or lower in a single ultrasound module. The transducer 210 may generate high-frequency ultrasound for a specific time and low-frequency ultrasound for another specific time. A description of structure and arrangement of the transducer 210 will be made later with reference to FIGS. 5 and 6.

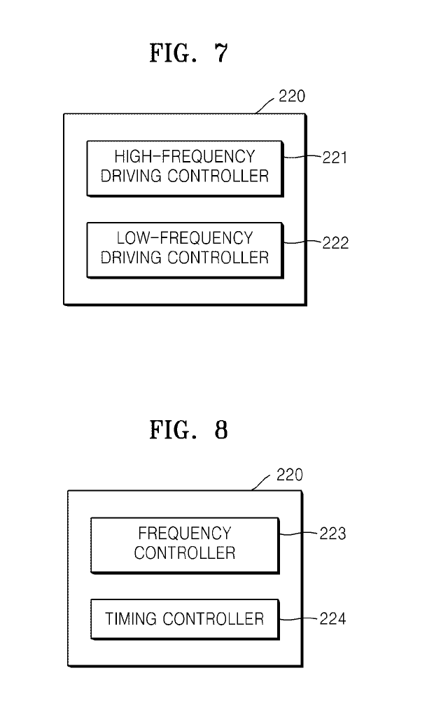

The controller 220 controls the transducer 210 to irradiate ultrasound to the treatment target portion 50 or a target portion of the skin layer 51. The controller 220 controls multiple elements (or ultrasound modules) that form the transducer 210 in such a way that each element is time-divided to irradiate ultrasound to a first element among the multiple elements at first timing and irradiate ultrasound to a second element at second timing. The controller 220 may separately control the first element that generates high-frequency ultrasound and the second element that generates low-frequency ultrasound among the multiple elements. The detailed structure of the controller 220 will be described later with reference to FIGS. 7 and 8.

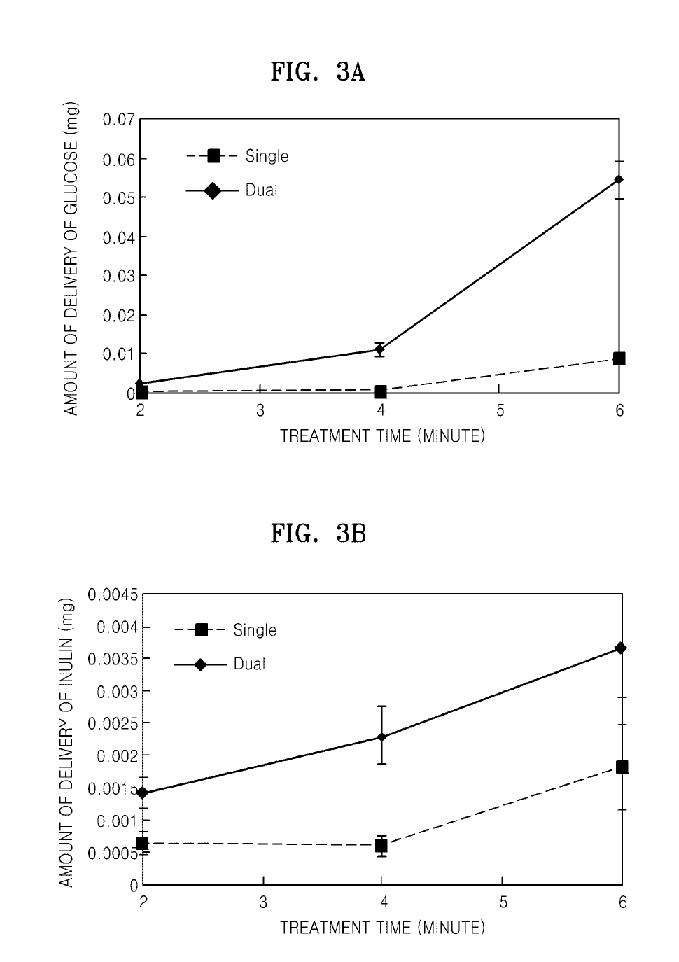

FIGS. 3A and 3B are diagrams illustrating examples for comparing a case where medication is delivered using one frequency with a case where medication is delivered using multiple frequencies. In FIGS. 3A and 3B, administration of medication using ultrasound of a single frequency and administration of medication using ultrasound of dual frequencies are compared to illustrate that the use of dual-frequency ultrasound is more effective.

Referring to FIG. 3A, a horizontal axis indicates a treatment time and a vertical axis indicates the amount of delivery of glucose. First, in ultrasound medication delivery therapy (phonophoresis) for up to four minutes, when a single frequency (high or low frequency) is used, the amount of delivery of glucose through the skin is about 0.001 mg. But when dual frequencies (high and low frequencies) are used, the amount of delivery of glucose through the skin is about 0.01 mg, which is about 10 times more efficient than the use of the single frequency. If the treatment time exceeds four minutes, an efficiency difference between the use of single-frequency ultrasound and the use of dual-frequency ultrasound is even widened.

Referring to FIG. 3B, a horizontal axis indicates a treatment time and a vertical axis indicates the amount of delivery of inulin. First, when single-frequency ultrasound is used up to four minutes, the amount of delivery of inulin through the skin is about 0.0006 mg, but when dual-frequency ultrasound is used, the amount of delivery of inulin through the skin is about 0.0023 mg, which is about 4 times more efficient than the use of single-frequency ultrasound.

Referring to FIGS. 3A and 3B, it can be seen that medication delivery ultrasound treatment using dual or multiple frequencies is more efficient than that using single-frequency ultrasound. Phonophoresis of high frequencies of about 1 through about 3 Mhz accelerates percutaneous penetration of low-molecular-weight medication, but as a molecular weight increases, the effect of acceleration of percutaneous penetration is reduced. Meanwhile, phonophoresis of low frequencies of about 20 through about 200 kHz has a great dynamic effect such as cavitation and has a deep penetration depth, such that high-molecular-weight medication, which may not easily penetrate through the skin using high-frequency phonophoresis, is also available in this frequency domain. But ultrasound energy in a continuous wave form may cause necrosis or injury of the skin tissue because low-frequency ultrasound has a great dynamic effect, such that low-frequency phonophoresis should be used with very low ultrasound energy.

The ultrasound system that delivers medication through the skin by using multiple frequencies may overcome disadvantages resulting from the use of a single frequency and efficiently deliver medication.

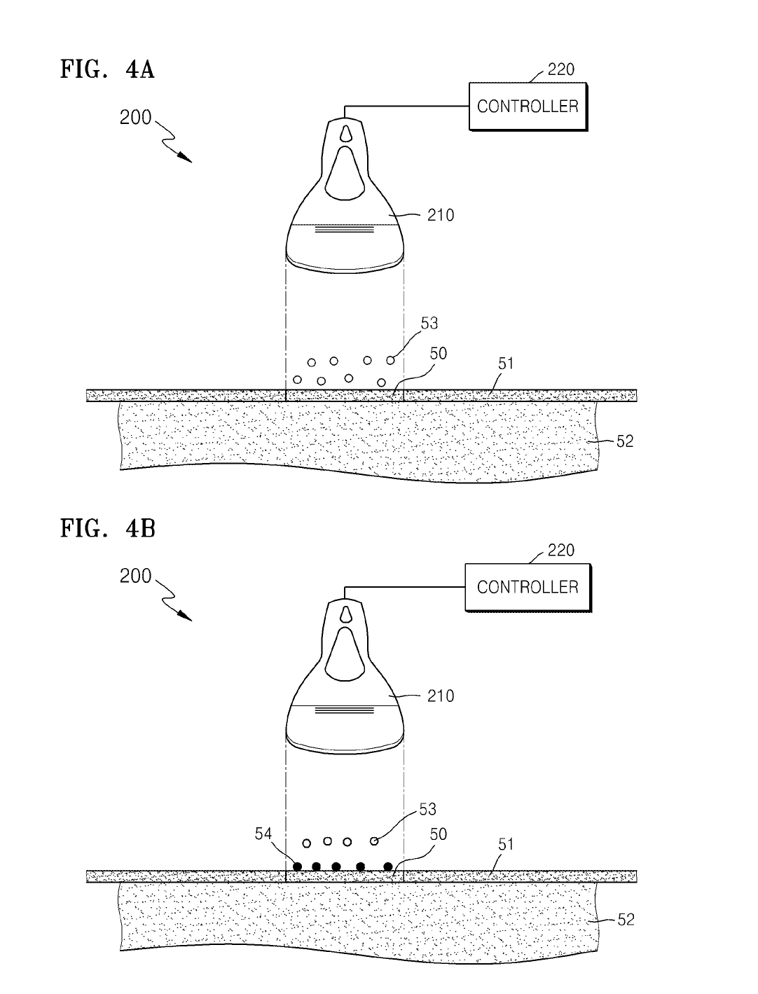

FIGS. 4A and 4B are diagrams illustrating examples of using an ultrasound system 200. The ultrasound system 200 delivers medication through the skin using dual frequencies. The ultrasound system 200 uses ultrasound of high frequencies of about 0.7 Mhz or higher and ultrasound of low frequencies of about 100 khz or lower together. Small bubbles in a cell membrane of the skin is generated (cativation) by irradiating the high-frequency ultrasound to the skin, and when low-frequency ultrasound is irradiated to the skin, it causes the small bubbles to collapse toward the skin. This improves the permeability of the cell membrane and thereby improves the efficiency of medication delivery through the skin. In this way, the permeability of medication through the cell membrane may be improved and a medication delivery time may also be reduced, thereby improving the efficiency of medication delivery.

Referring to FIG. 4A, the controller 220 controls the transducer 210 to irradiate high-frequency ultrasound to the target portion 50 of the skin 51. If the high-frequency ultrasound is irradiated to the transdermal tissue of the target portion 50, small bubbles 53 are generated on the skin tissue and are cavitated. That is, as moisture in the cell of the transdermal tissue is transformed into gas, micro bubbles are generated and these micro bubbles grow enough to cause resonance.

Referring to FIG. 4B, the controller 220 controls the transducer 210 to irradiate low-frequency ultrasound to the target portion 50 of the skin 51. If low-frequency ultrasound is irradiated to the cavitated bubbles in the target portion 50, then the bubbles are collapsed or are burst (indicated by 54) toward the target portion 50 of the skin 51, injuring the tissues of the target portion 50. Therefore, medication delivery occurs through the injured tissues, such that the permeability of medication into the body 52 may be improved.

The ultrasound system 200 according to an embodiment of the present disclosure may irradiate ultrasound of high frequencies of about 0.7 Mhz or higher and ultrasound of low frequencies of about 100 khz or lower through one transducer 210. A time for irradiation of the high-frequency ultrasound and a time for irradiation of the low-frequency ultrasound may be arbitrarily set according to one or more of characteristics of the ultrasound system 200, a type of delivery medication, and a molecular weight of the medication. Before the high-frequency ultrasound shown in FIG. 4A is irradiated, medication to be delivered to the target portion 50 may be injected in advance. Although not shown in the drawings, the ultrasound system 200 may also include other general components. For example, a medication storing unit or a medication injecting unit may be included in the ultrasound system 200.

FIG. 5 is a diagram illustrating an example for describing a method of controlling the ultrasound system 200, and FIG. 8 is a diagram illustrating an example of the controller 220 for implementing the method of controlling the ultrasound system 200.

Referring to FIGS. 2, 5, and 8, the transducer 210 is formed of a material having a broad frequency band. For example, the transducer 210 may be formed of single crystal or capacitive micromachined ultrasound transducer (cMUT). In the cMUT, a ceramic vibrator in the form of a thin drumhead may be suspended on a silicon wafer. The diameter of the vibrator is about 30 μm, and its thickness is about 3000 Å through about 7000 Å, in which an interval between the wafer and the vibrator is merely about several thousands of Å. If an electric signal is applied between the wafer and the vibrator, an electrostatic force is applied to the vibrator and ultrasound is generated from the electrostatic force. Since the intensity of ultrasound generated in one vibrator is not sufficiently high, generally several thousands of identical vibrators are vibrated at the same time. The cMUT has a broadband feature and may generate ultrasound of high frequencies of about 0.7 MHz or higher and ultrasound of low frequencies of about 100 kHz or lower.

As shown in FIG. 8, the controller 220 includes a frequency controller 223 and a timing controller 224. Since the transducer 210 has a broad bandwidth, the controller 220 selects high-frequency ultrasound and low-frequency ultrasound under control of the frequency controller 223. The timing controller 224 generates high-frequency ultrasound and low-frequency ultrasound at particular timings. As shown in FIG. 5, the high-frequency ultrasound is irradiated after the low-frequency ultrasound is irradiated. That is, when one transducer has a broad bandwidth, irradiation timings are time-divided to alternately irradiate the low-frequency ultrasound and the high-frequency ultrasound with one transducer. By using the dual frequencies, such a scheme may be applied to the ultrasound system to deliver medication through the skin.

The skin's tissue is cavitated after high-frequency ultrasound is selected and then irradiated, low-frequency ultrasound is selected and irradiated to collapse the cavitated tissue with respect to the skin, injuring the skin tissue. Thus, medication delivery may be efficiently achieved through the injured skin. In the timing diagram shown in FIG. 5, a low frequency 510 and a high frequency 520 are irradiated at the same time intervals, but they may be irradiated with variable time intervals based on a type or a molecular weight of medication to be delivered or a type of medication treatment.

FIGS. 6A through 6D are diagrams illustrating examples of a transducer for using multiple frequencies, and FIG. 7 is a diagram illustrating an example of the controller 220 which controls the transducer for using multiple frequencies. In the transducer shown in FIGS. 6A through 6D, elements for generating high-frequency ultrasound and elements for generating low-frequency ultrasound are divided and elements may be implemented in various forms and with various arrangements in the single transducer. FIGS. 5 and 8, describe one transducer that generates ultrasound having a broad bandwidth. FIGS. 6A through 7, describe an element for generating low-frequency ultrasound and an element for generating high-frequency ultrasound disposed in a single transducer.

FIG. 6A illustrates an example of a transducer where a high-frequency ultrasound module 600 and a low-frequency ultrasound module 610 are arranged in concentric circular form. The ultrasound module should be understood as an element included in a single transducer or a piezoelectric material. FIG. 6B illustrates an example of a transducer in which the high-frequency ultrasound module 600 and the low-frequency ultrasound module 610 are arranged in repetitive concentric circular form. FIG. 6C illustrates an example of a transducer in which the high-frequency ultrasound module 600 and the low-frequency ultrasound module 610 are randomly arranged. FIG. 6D illustrates an example of a transducer in which the high-frequency ultrasound module 600 and the low-frequency ultrasound module 610 are arranged in a quadrilateral form.

In the examples described with reference to FIGS. 6A and 6B, a high-frequency ultrasound module and a low-frequency ultrasound module have been included to use multiple frequencies, but a transducer having a single element may also be used. For example, by using an element that generates low-frequency ultrasound, ultrasound of a high frequency that is about 3 times a low frequency may be generated. One element may generate multiple frequencies, both high and low. This feature uses the high output efficiency of the single element at harmonic frequency. Therefore, by outputting a frequency that is three times a low frequency, which is the first harmonic frequency, high-frequency ultrasound may be output. After high-frequency ultrasound is irradiated as shown in FIG. 5, a time-division scheme may be used to output low-frequency ultrasound.

As shown in FIG. 7, the controller 220 includes a high-frequency driving controller 221 and a low-frequency driving controller 222. The high-frequency driving controller 221 drives the high-frequency ultrasound module 600 to irradiate high-frequency ultrasound, thus cativating the skin's tissue. The low-frequency driving controller 222 drives the low-frequency ultrasound module 610 to irradiate low-frequency ultrasound, thereby collapsing the cavitated tissue with respect to the skin and injuring the skin's tissue. Thus, medication may be efficiently delivered through the injured skin.

While some combinations, forms, and arrangements of a high-frequency ultrasound module and a low-frequency ultrasound module are shown in FIGS. 6A through 6D, other forms may also be possible. Although it has been described with reference to FIGS. 5 through 6D that high-frequency ultrasound and low-frequency ultrasound may be irradiated in a time-division or space-division scheme, both the time-division scheme and the space-division scheme may be used together.

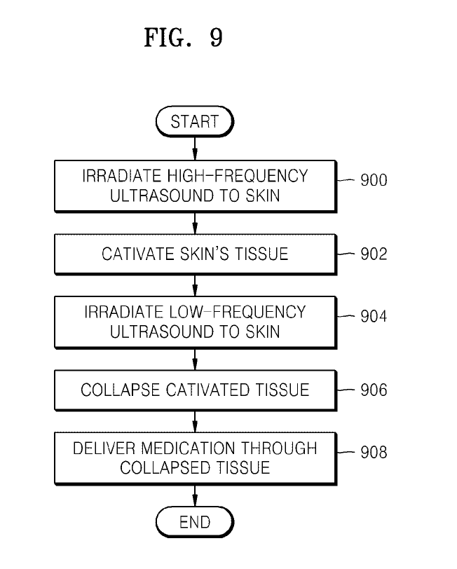

FIG. 9 is a diagram illustrating an example for describing a method of controlling an ultrasound system. The operations in FIG. 9 may be performed in the sequence and manner as shown, although the order of some operations may be changed or some of the operations omitted without departing from the spirit and scope of the illustrative examples described. Many of the operations shown in FIG. 9 may be performed in parallel or concurrently. The description of FIGS. 1-8 is also applicable to FIG. 9, and thus will not be repeated here. Referring to FIG. 9, in step 900, high-frequency ultrasound is irradiated to the skin. A high-frequency range represents a frequency range of 0.7 MHz or higher.

In step 902, the skin's tissue is cavitated. In step 902, if the skin's tissue is exposed to high-frequency ultrasound, moisture in the cell is transformed into gas, generating micro bubbles which then grow to cause resonance (cavitation).

In step 904, low-frequency ultrasound is irradiated to the skin. A low-frequency range represents a frequency range of 100 kHz or lower.

In step 906, the cavitated tissue is collapsed. If the low-frequency ultrasound is irradiated to the cavitated tissue, the micro bubbles are collapsed toward the skin and at the same time, generate shock waves of a high pressure, thus injuring peripheral tissues. In step 908, medication is delivered through the collapsing tissue.

According to an aspect, an ultrasound system irradiates high-frequency ultrasound and low-frequency ultrasound to the skin in a time-division or space-division manner, thereby improving the efficiency of medication delivery through the skin.

The processes, functions, and methods described above including a method for beamforming can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, WiFi, etc.). In addition, functional programs, codes, and code segments for accomplishing the example disclosed herein can be construed by programmers skilled in the art based on the flow diagrams and block diagrams of the figures and their corresponding descriptions as provided herein.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method to deliver medication through skin comprising:
irradiating the skin with ultrasound having a first frequency to cavitate a skin tissue;
irradiating the skin with ultrasound having a second frequency, which is lower than the first frequency, to collapse the cavitated tissue; and
delivering the medication through the collapsed tissue,
wherein a single transducer is configured to produce the ultrasound having the first frequency and the ultrasound having the second frequency.

2. The method of claim 1, wherein the single transducer comprises multiple elements, a first element that is configured to irradiate the ultrasound having the first frequency and a second element that is configured to irradiate the ultrasound having the second frequency.

3. The method of claim 1, wherein the first frequency is at least three times the second frequency.

4. The method of claim 1, wherein the single transducer is configured to irradiate the skin with the ultrasound having the first frequency and the ultrasound having the second frequency in a time-division manner.

5. The method of claim 1, wherein the single transducer is configured to irradiate the skin with the ultrasound having the first frequency and the ultrasound having the second frequency in a space-division manner.

6. The method of claim 4, wherein the single transducer is formed of a crystal material.

7. The method of claim 4, wherein the single transducer is a capacitive micromachined ultrasound transducer (cMUT).

8. The method of claim 2, wherein each of the multiple elements are formed of a piezoelectric material.

9. The method of claim 2, wherein the first element and the second element are arranged in a circular manner in the single transducer.

10. The method of claim 2, wherein the first element and the second element are arranged at random in the single transducer.

11. The method of claim 2, wherein the first element and the second element are arranged in a quadrilateral array in the single transducer.

12. The method of claim 2, wherein a phase-array scheme is used to change a position of irradiation without changing the position of the single transducer.

13. The method of claim 1, wherein a time of irradiating the skin with ultrasound having a second frequency and a time of irradiating the skin with ultrasound having a first frequency is based on at least one of characteristics of the ultrasound system, type of medical treatment, medication, and a molecular weight of the medication.

14. An ultrasound system to deliver medication through skin comprising:
a first ultrasound module configured to irradiate a skin with ultrasound having a first frequency to cavitate the skin tissue;
a second ultrasound module configured to irradiate the skin with ultrasound having a second frequency, which is lower than the first frequency, to collapse the cavitated tissue; and
a controller configured to drive the first ultrasound module and the second ultrasound module,
wherein the first ultrasound module and the second ultrasound module are disposed in a single transducer.

15. The ultrasound system of claim 14, wherein the controller drives the first ultrasound module and the second ultrasound module in a time-division manner.

16. The ultrasound system of claim 15, wherein the first ultrasound module and the second ultrasound module are formed of a crystal material.

17. The ultrasound system of claim 15, wherein the first ultrasound module and the second ultrasound module are capacitive micromachined ultrasound transducer (cMUT).

18. The ultrasound system of claim 15, wherein the first ultrasound module and the second ultrasound module are formed of a piezoelectric material.

19. The ultrasound system of claim 15, wherein the first ultrasound module and the second ultrasound module are arranged in a circular manner in the single transducer.

20. The ultrasound system of claim 15, wherein the first ultrasound module and the second ultrasound module are arranged at random in the single transducer.

21. The ultrasound system of claim 15, wherein the first ultrasound module and the second ultrasound module are arranged in a quadrilateral array in the single transducer.

22. The ultrasound system of claim 14, wherein the controller comprises a first driving controller and a second driving controller, wherein the first driving controller is configured to drive the first ultrasound module and the second driving controller is configured to drive the second ultrasound module.

23. An ultrasound system to deliver medication through skin, the ultrasound system comprising:
a transducer comprising a first ultrasound module configured to irradiate ultrasound having a first frequency and a second ultrasound module configured to irradiate ultrasound having a second frequency, which is lower than the first frequency; and
a controller configured to control the transducer to irradiate a skin with the ultrasound having the first frequency and the ultrasound having the second frequency.

24. The ultrasound system of claim 23, wherein the controller is configured to control the first ultrasound module and the second ultrasound module in a time-division manner to alternately irradiate the skin.
